(19) United States
(12) Patent Application Publication
(10) Pub. No.: US 2006/0282103 A1
(43) Pub. Date: Dec. 14, 2006

(54) Surgical planar plug

(76) Inventors: Helmut Fricke, Meinersen (DE); Hanngorg Zimmermann, Rosstal (DE)

Correspondence Address: MCGLEW & TUTTLE, PC, P.O. BOX 9227, SCARBOROUGH STATION, SCARBOROUGH, NY, 10510-9227, US

(21) Appl. No.: 10/554,715

(22) PCT Filed: Apr. 20, 2004
(86) PCT No.: PCT/EP04/04169

§ 371 Date: Oct. 27, 2005

(30) Foreign Application Priority Data
Apr. 28, 2003 (DE) 203 06 635.9

(51) Int. Cl. A61B 17/08

(52) U.S. Cl. 606/151

(57) ABSTRACT

A surgical flat implant for preventing tissue-to-tissue adhesion in operated areas, in particular for post-operative repair in pericardial, peritoneal and gynaecological surgery, comprises at least one layer (1) of a thin, bioresorbable, smooth film and a stabilizing layer in form of a reinforcing mesh (2) of plastic material which is provided with a metal-containing, biocompatible, continuous coating (4).

The invention relates to a surgical flat implant for preventing tissue-to-tissue adhesion in operated areas, in particular for post-operative repair in pericardial, peritonial or gynaecological surgery, according to the preamble of claim 1.

Flat implants of the generic type have been known in surgery, comprising a layer of a thin, bioresorbable, smooth film; they function as a barrier to the mentioned tissue-to-tissue adhesion.

Tissue-to-tissue adhesion consists in fibrous ligaments forming abnormal connections between two layers of body tissue that are normally separated. Adhesion of that type or so-called scar tissue can lead to lots of complications, such as severe abdominal pain, infertility or intestinal obstruction. Tissue-to-tissue adhesion is based on cell reaction after a traumatic tissue injury as it takes place during surgery. After the trauma, a fibrin matrix starts forming in the tissue. Fibroplasts, fibrin filaments and histamines proliferate. Gradually fibrin ligaments form between two layers of tissue, with the resulting adhesive forces contracting the surrounding tissue, which produces opposite effects. On the whole, considerable interference with the layered body structure takes place in operated areas.

So as to avoid the above problems, bioresorbable barrier films are inserted in the operated areas, thus precluding any adhesion and providing surgical divisions for any possible posterior surgery.

The barrier films are ultra-thin and very easily deformable, excellently conforming to the surrounding body. As a rule, this effect is desired. However, there are cases where higher stability is needed, with additional stabilization of the operated area being desired for example for the support of scars. In these cases, the film does not offer much in terms of support.

It is an object of the invention to embody a flat surgical implant of increased mechanical stability which prevents tissue-to-tissue adhesions in operated areas.

This object is attained by the features specified in the characterizing part of claim 1. Accordingly, a bioresorbable layer of film is joined to a stabilizing layer in the form of reinforcing mesh of plastic material that is provided with a metal-containing, continuous coating tolerated by the human body. Reinforcing mesh of the species has been known per se as separate products in the field of medical engineering, namely as so-called hernia mesh fabric used for stabilization of the abdominal wall in inguinal hernia surgery.

The flat implant according to the invention comprises a composite structure, with each of the two layers fulfilling specific tasks. The bioresorbable film precludes tissue-to-tissue adhesion during a post-surgical phase and decomposes gradually. The reinforcing mesh stabilizes the operated area from the very beginning, but stays in the body after degradation of the barrier film, durably continuing the stabilizing task.

With the reinforcing mesh remaining in the body, the metal-containing, biocompatible, continuous coating gains special importance.

Being produced preferably on the basis of titanium-containing compounds of a thickness of <2 μm, preferably of 5 to 700 nm, this coating fulfils a lot of tasks. For example, as a result of its continuity, there is no longer any direct contact of the basic plastic material of the reinforcing mesh with body tissue, the mesh being felt by the body to consist continuously of preferably titanium-containing material. Any reactions of rejection in the form of encapsulation of the mesh are not set in train. Evidently, the metal-containing coating constitutes a diffusion barrier to the plasticizer molecules in the plastic material of the mesh, precluding the plasticizer from diffusing. This minimizes any embrittlement of the mesh.

The metal-containing coating preferably consists of a compound of the formula TiₐO_{b}C_{c}, with
a=0.025 to 0.9,
b=0.025 to 0.7 and
c=0.2 to 0.9
   applying.

Optionally, the titanium constituents can be replaced by tantalum, niobium, silver, zirconium and hafnium. Nitrogen and boron may be further elements in the compound.

The plastic material of the reinforcing mesh preferably consists of polypropylene, polyurethane, polypolyester or PTFE. The bioresorbable film is produced on the basis of polylactate.

Joining the film layer to the stabilizing layer in the form of the reinforcing mesh may for example be implemented by means of spot gluing at the points where the mesh and the film intersect, in which case use is preferably made of a bioresorbable or at least biocompatible adhesive, such as a fibrin adhesive. Mechanical connection by the aid of knotted filaments may consitute an alternative, in which case the knotted filaments themselves, like the reinforcing mesh, are provided with the continuous, biocompatible, metal-containing coating.

Finally, a hemostyptic layer for hematostatic-agent release can be incorporated as a third component into the compound system.

Further features, details and advantages of the invention will become apparent from the ensuing description of exemplary embodiments, taken in conjunction with the drawings, in which

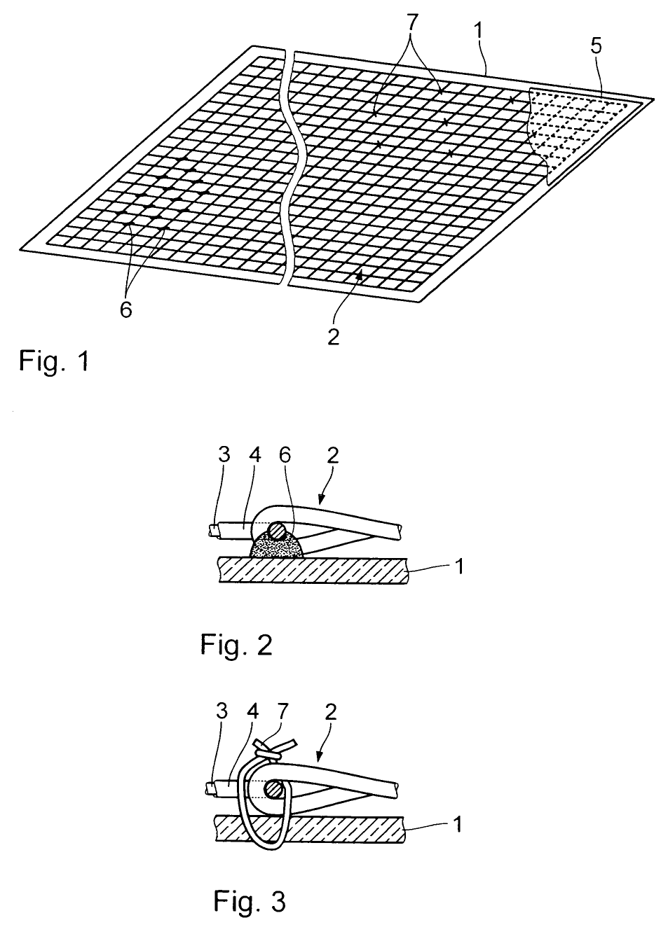

FIG. 1 is a diagrammatic perspective view of a surgical flat implant;

and

FIGS. 2 and 3 are enlarged detailed sectional views crosswise of the plane of the layers of two varying embodiments of the flat implant.

As seen in FIG. 1, the flat implant is comprised of a thin, bioresorbable, smooth film layer 1, which is produced on the basis of a polylactate. The thickness thereof is for instance in the range of 0.02 or 0.05 mm. The dimensions of the area range between 70 and 200 mm in length and 50 and 130 mm in width. Films of that type are commercialized for example under the brand “Surgi wrap”® by the company MacroPore Biosurgery, Inc. of San Diego, Calif., U.S.A. After insertion of the flat implant into the human body, the film layer 1 decomposes because of is bioresorbable properties typically within three months.

A reinforcing mesh 2 is applied to the film layer 1; it is knitted from a synthetic filament 3, for example of polypropylene, polyurethane, polyester or PTFE. As seen in FIGS. 2 and 3, this synthetic filament 3 is externally provided with a continuous biocompatible coating 4 of titanium-containing material of the formula TiₐO_{b}C_{c}.

The constituent ranges a, b and c correspond to those mentioned in the introductory part. Such a coating has proved absolutely biocompatible, its continuity having the effect that the entire plastic core of the filament 3 will not longer be perceived as such by the human body. Consequently, the bio-compatibility is comparable to implants which are completely made from a titanium alloy and which are widely accepted in medical engineering.

Preferred thicknesses of the coating 4 are in the range of 5 to 700 nm, with coating thicknesses of approximately 50 nm having proved to possess special adhesive and frictional strength on the one hand and to be sufficiently flexible and ductile on the other, the coating thus being able without any damages to participate in any stretching of, and strain on, the filament and, consequently, the reinforcing mesh.

Titanium-containing coatings of that type and the technique of applying them on flexible plastic substrates are fundamentally known from the prior art, for example from EP 0 897 997 B1.

A reinforcing mesh 2 as such and the implementation thereof in the form of knitted fabric of a metal-coated synthetic filament 3 have been described in detail in Applicant's prior German patent application 102 21 320.8.

As roughly outlined in FIG. 1, a hemostyptic layer 5 can be applied to the outside of the flat implant i.e., on the reinforcing mesh 2, as another component of the flat compound (see FIG. 1, right outward corner). This hemostyptic layer comprises a hematostatic agent which can gradually be administered to the body for example by resorption of the layer 5.

As for the layer system, it must be emphasized that several layers of reinforcing mesh and film may be provided in varying layer structures. For example, a central reinforcing mesh can be combined with two bilateral layers of film 1 or, vice versa, a film layer 1 can combine with two bilateral reinforcing meshes 2. Sandwich structures comprised of several film layers 1 and reinforcing meshes 2 in alternating placement are conceivable, depending on the purpose.

Various techniques can be used to connect the individual layers, which is roughly outlined in FIG. 1. Glued spots 6 are perceptible in FIG. 1 on the left and in FIG. 2; they are located at the points of intersection of the reinforcing mesh. The glued spots 6 may for example be drops of a biocompatible, fibrin adhesive.

An alternate mode of connection is seen in FIG. 1 on the right and in FIG. 3, namely a spotwise connection of the reinforcing mesh 2 with the film layer 1 by means of knotted filaments 7 which are led through the film layer 1 at points of intersection of the reinforcing mesh 2 in the way of basting stitches and are then knotted. The knotted filaments 7 themselves are provided with the described, biocompatible, metal-containing and titanium-based coating so that they do not produce any reactions of rejection by the human body.

1. A surgical implant for preventing tissue-to-tissue adhesion in operated areas, in particular for post-operative repair in pericardial, peritoneal or gynaecological surgery, comprising
at least one film layer of a thin, bioresorbable, smooth film, and
a stabilizing layer in form of a reinforcing mesh of plastic material which is joined to the film layer and which is provided with a metal-containing, biocompatible, continuous coating.

2. A surgical flat implant according to claim 1, wherein the coating is a titanium-containing coating of a thickness of less than 2 μm, preferably of 5 to 700 nm.

3. A surgical flat implant according to claim 2, wherein the coating*) comprises a compound of a formula
TiₐO_{b}C_{c},
with a=0.025 to 0.9,
b=0.025 to 0.7 and
c=0.2 to 0.9
applying.

4. A surgical flat implant according to claim 1, wherein the reinforcing mesh consists of polypropylene, polyurethane, polyester or PTFE.

5. A surgical flat implant according to claim 1, wherein the bioresorbable film layer consists of a material selected from a polylactate.

6. A surgical flat implant according to claim 1, wherein the reinforcing mesh is joined to the film layer by glued spots.

7. A surgical flat implant according to wherein the reinforcing mesh is joined to the film layer by spots by means of knotted filament which are also provided with said continuous, biocompatible, metal-containing coating.

8. A surgical flat implant according to claim 1, wherein a hemostyptic layer for hematostatic-agent release is provided preferably on an outside of the flat implant.
